(19) United States
(12) Patent Application Publication
(10) Pub. No.: US 2009/0043383 A1
(43) Pub. Date: Feb. 12, 2009

(54) GENETICALLY MODIFIED HEART VALVE XENOGRAFTS

(75) Inventors: Christopher G.A. McGregor, Rochester, MN (US); Guerard W. Byrne, Rochester, MN (US); William R. Davies, Cambridgeshire (GB); John S. Logan, Robbinsville, NJ (US)

Correspondence Address: QUARLES & BRADY LLP, 411 E. WISCONSIN AVENUE, SUITE 2040, MILWAUKEE, WI, 53202-4497, US

(73) Assignee: MAYO FOUNDATION FOR MEDICAL EDUCATION AND RESEARCH, Rochester, MN (US)

(21) Appl. No.: 10/594,944

(22) PCT Filed: Mar. 28, 2005
(86) PCT No.: PCT/US2005/010291

§ 371 Date: Oct. 8, 2008

Related U.S. Application Data
(60) Provisional application No. 60/557,238, filed on Mar. 29, 2004.

(51) Int. Cl. A61F 2/24

(52) U.S. Cl. 623/2.42; 600/36

(57) ABSTRACT

The invention relates to heart valve xenografts from transgenic pigs having a disruption of an α1-3 galactosyl transferase nucleic acid sequence and use of the xenografts for treating a patient.

### CROSS-REFERENCE TO RELATED APPLICATION

This application claims priority to U.S. Ser. No. 60/557,238, filed Mar. 29, 2004, incorporated by reference herein.

### STATEMENT REGARDING FEDERALLY SPONSORED RESEARCH OR DEVELOPMENT

### BACKGROUND OF THE INVENTION

This invention relates to heart valve xenografts, and more particularly to heart valve xenografts from animals having a disruption in the α1-3 galactosyl transferase nucleic acid sequence.

Prosthetic heart valves are used to replace damaged or diseased heart valves, including the aortic, mitral (bicuspid), tricuspid, and pulmonary heart valves. There are two basic types of prosthetic heart valves, mechanical and tissue-type valves. Mechanical heart valves use a pivoting mechanical closure to provide unidirectional blood flow, while tissue type valves are made from natural tissue valve leaflets. Mechanical valves are made of pyrolytic carbon, and although they do not wear out, they require life-long anticoagulation, with an increased incidence of thrombotic and hemorrhagic complications.

Tissue valves resemble native valves, and do not require life-long anticoagulation, but they wear out over time (in general after about 10 years). Much of the structure and many of the properties of original heart valves can be retained in transplants through use of heterograft or xenograft materials, that is, a heart valve from a different species than the graft recipient. Once implanted in an individual, a xenograft provokes hyperacute rejection (HAR), which occurs within minutes to three hours of implantation. HAR can be overcome by a number of methodologies. If HAR is avoided, the organs can be rejected within a few days to weeks, even in the presence of a regimen of immunosuppressive agents that are effective at preventing allograft rejection. Xenografts can be chemically treated to reduce immunogenicity prior to implantation into a recipient or subjected to various physical treatments in preparation for implantation.

### BRIEF SUMMARY OF THE INVENTION

The invention is based on the identification that porcine heart valves and commercially available porcine heart valve xenografts are positive for galactose α1,3 galactose β1,4 N-acetylglucosamine trisaccharide (Gal α1-3Galβ1-4GlcNac), i.e., the Gal or α-gal antigen. Use of heart valve xenografts from transgenic pigs having a disruption of an α1-3 galactosyl transferase nucleic acid sequence and that have reduced or no detectable Gal antigen, can reduce immunogenicity of the xenograft upon implantation and prolong durability of the xenograft.

In one aspect, the invention features a method of treating a patient. The method includes implanting into the patient a porcine heart valve xenograft; wherein cells of the xenograft contain a disruption of the α1-3 galactosyl transferase nucleic acid sequence. The heart valve xenograft can be a tricuspid valve or a portion thereof, a mitral valve or a portion thereof, an aortic valve or a portion thereof, or a pulmonary valve or a portion thereof. The heart valve xenograft can be pericardial tissue.

In another aspect, the invention features an article of manufacture that includes a porcine heart valve xenograft and a storage solution, wherein cells of the xenograft contain a disruption of the α1-3 galactosyl transferase nucleic acid sequence. The storage solution can be saline, a tissue preservative, or a cryoprotectant. The cryoprotectant can be dimethylsulfoxide, glycerol, albumin, monosaccharides, disaccharides, or serum.

The invention also features a method of preparing a xenograft heart valve for implantation in a human. The method includes providing a xenograft from a pig, wherein the xenograft includes a portion of a heart valve, wherein the pig's genome includes a disruption of an α1-3 galactosyl transferase nucleic acid sequence, the disruption resulting in endothelial cells of the pig having reduced or no detectable expression of Gal α1-3Galβ1-4GlcNac on their surface relative to cells of a control pig; and contacting the xenograft with a fixative. The fixative can be selected from the group consisting of gluteraldehyde, formaldehyde, adipic dialdehyde, an aliphatic diamine, an aromatic diamine, a carbodiimide, and a diisocyanate. Gluteraldehyde is a particularly useful fixative. The method further can include subjecting the xenograft to a freeze/thaw cycle. The method further can include contacting the xenograft with an agent selected from the group consisting of an anti-calcification agent, an anti-thrombotic agent, an antibiotic, and a growth factor. The method further can include sterilizing the xenograft.

In another aspect, the invention features an article of manufacture that includes a heart valve xenograft from a pig, wherein the pig's genome includes a disruption of an α1-3 galactosyl transferase nucleic acid sequence, the disruption resulting in endothelial cells of the pig having reduced or no detectable expression of Gal α1-3Galβ1-4GlcNac on their surface relative to cells of a control pig. The xenograft can be attached to a stent.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE SEVERAL VIEWS OF THE DRAWINGS

FIG. 1 is a photomicrograph of GSIB4 (Griffonia simplicifolia IB4 lectin) staining of normal (GT^{+/+}) pig cardiac tissue. FIG. 1A: Staining with GSIB4-HRP (horse radish peroxidase conjugated GSIB4 lectin) detects widespread expression of the α-gal antigen on endothelium of microvascular blood vessels (arrows). FIG. 1B: Lectin staining is blocked by competition with 10 mM α-gal trisaccharide.

FIG. 2 is a photomicrograph of GSIB4 staining of normal (GT^{+/+}) cardiac valve tissue. FIG. 2A: Staining with GSIB4-HRP. FIG. 2B: Competition with 10 mM α-gal trisaccharide.

FIG. 3 is a photomicrograph of GSIB4 staining of normal (GT^{+/+}) cardiac valve tissue. FIG. 3A: Hematoxylin and eosin staining. FIG. 3B: Staining with GSIB4-HRP. FIG. 3C: Competition with 10 mM α-gal trisaccharide.

FIG. 4 is a photomicrograph of GSIB4 staining of eight commercial porcine bioprosthetic valve devices. FIG. 4A: Hematoxylin and eosin staining. FIG. 4B: Staining with GSIB4-HRP. FIG. 4C: Competition with 10 mM α-gal trisaccharide.

FIG. 5 is a photomicrograph of comparison of GSIB4 staining of normal GT^{+/+} (A-C) and α-gal deficient GT⁻⁻ (D-F) cardiac valves. FIGS. 5A and D: Hematoxylin and eosin staining. FIGS. 5B and E: Staining with GSIB4-HRP. FIGS. 5C and F: Competition with 10 mM α-gal trisaccharide.

FIG. 6 is a photomicrograph of GSIB4 staining of α-gal deficient (GT^{-/-}) cardiac mitral valve tissue. FIG. 6A: hematoxylin and eosin staining. FIG. 6B: Staining with GSIB4-HRP. FIG. 6C: Competition with 10 mM α-gal trisaccharide.

### DETAILED DESCRIPTION OF THE INVENTION

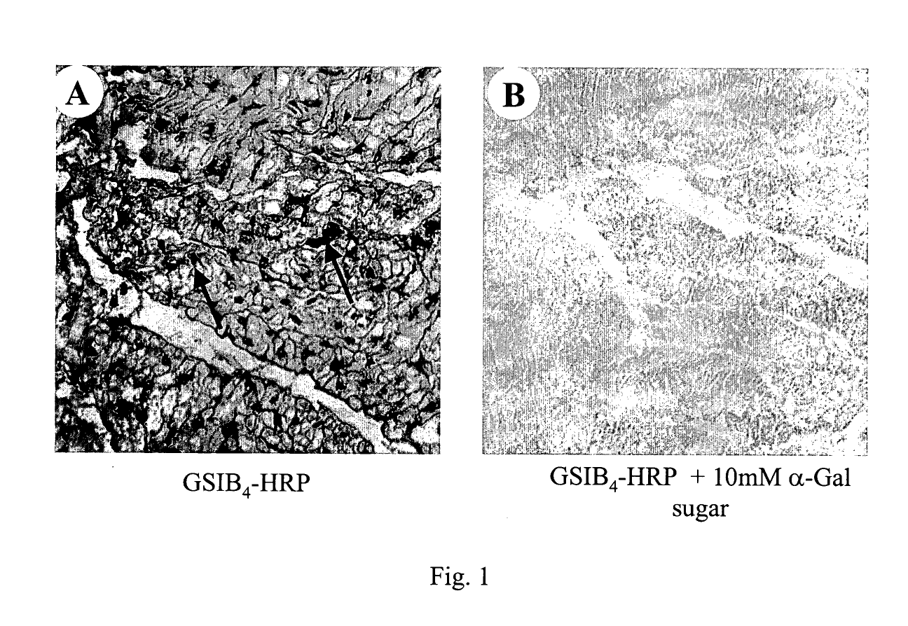

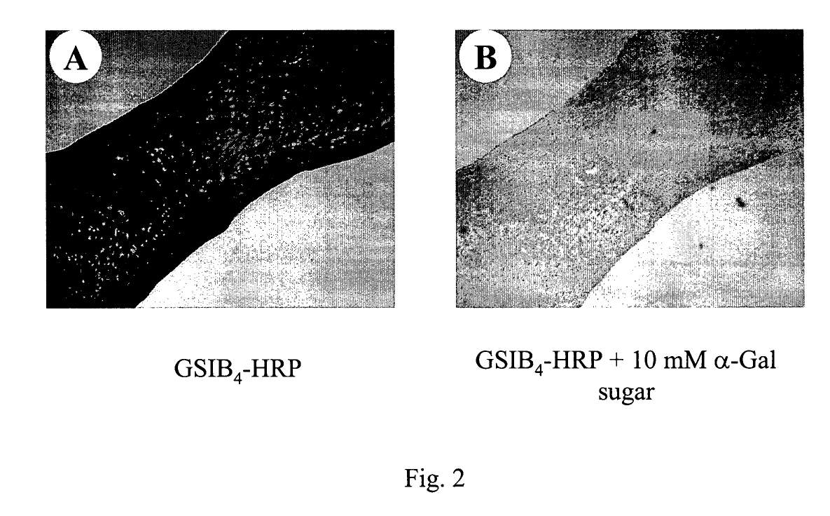

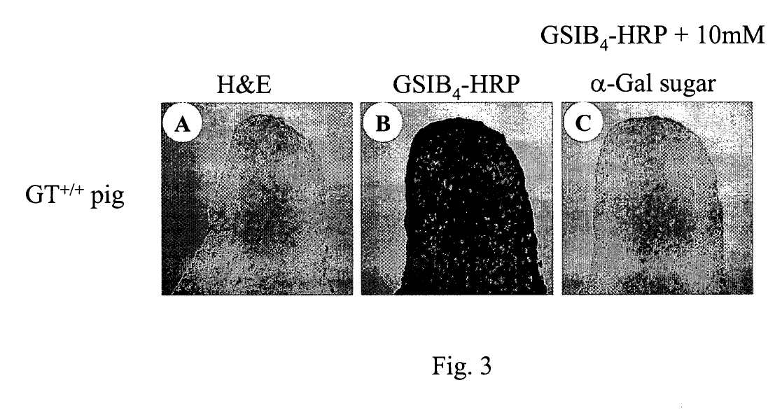

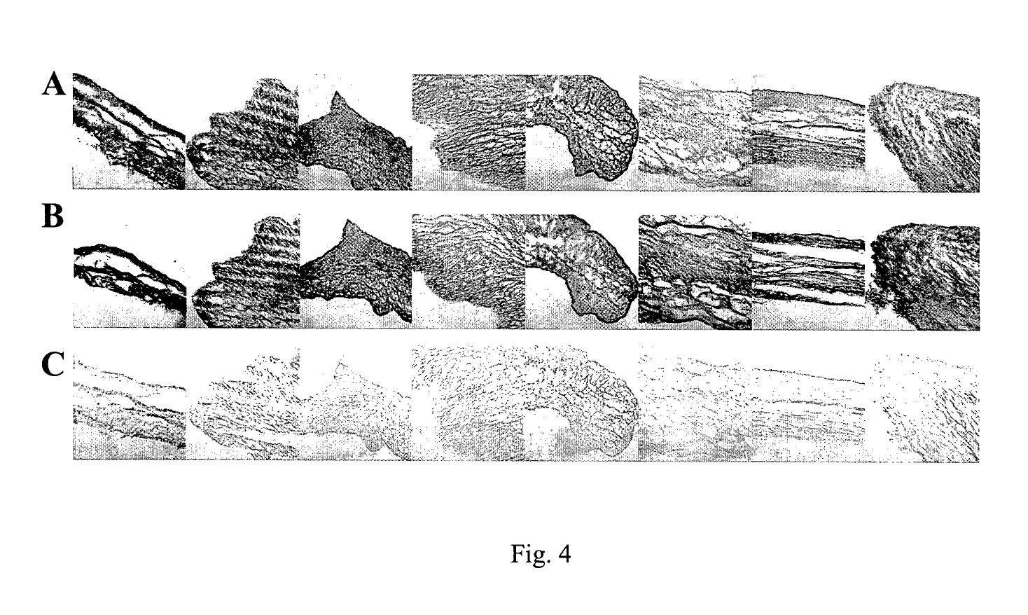

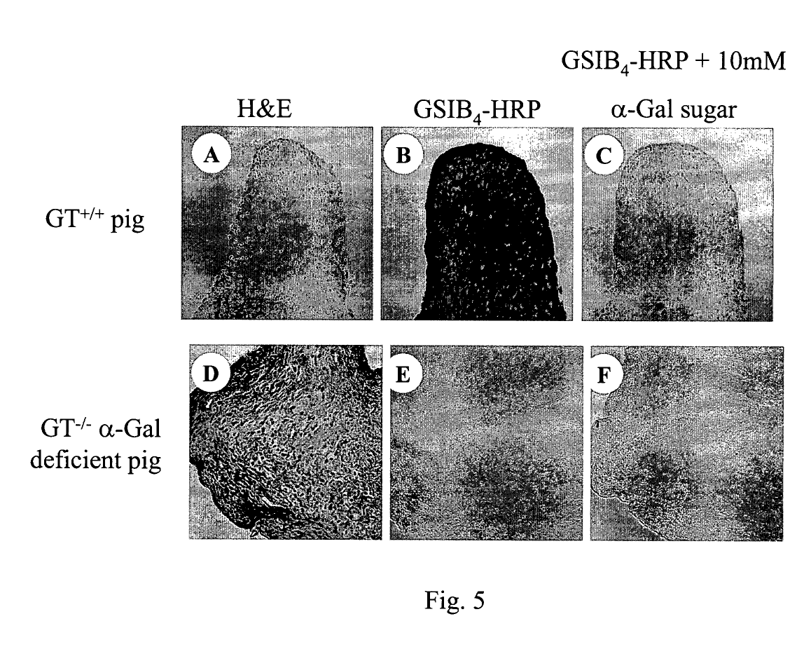

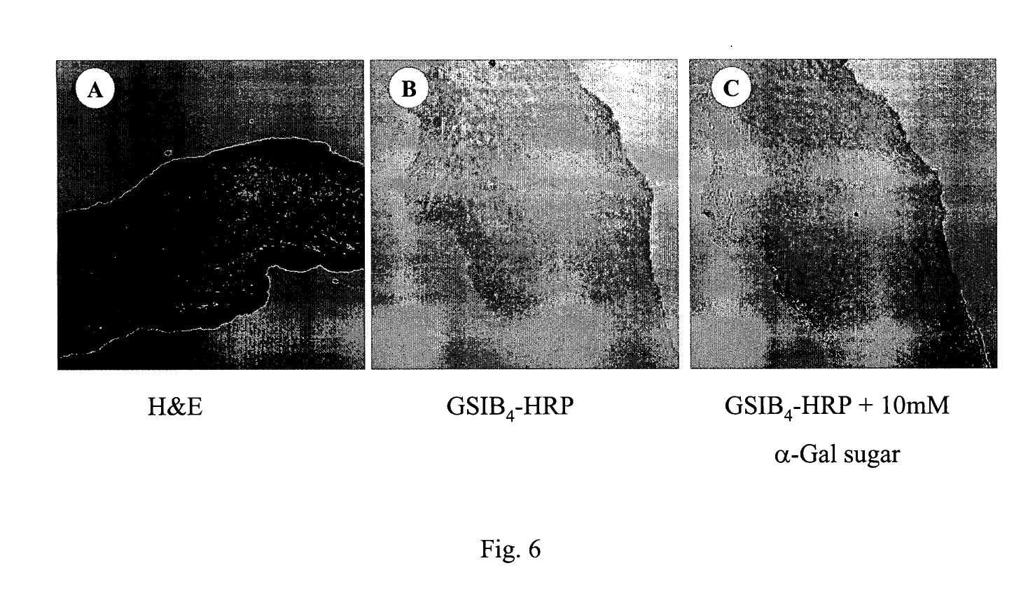

In general, the invention provides heart valve xenografts from transgenic pigs having a disruption of an α1-3 galactosyl transferase nucleic acid sequence. Disruption of the α1-3 galactosyl transferase nucleic acid sequence results in the transgenic pig having reduced or no detectable α1-3 galactosyl transferase activity, and consequently, endothelial cells from such pigs have reduced no detectable expression of Gal α1-3Galβ1-4GlcNac (i.e., the Gal antigen) on their surface (FIGS. 5 and 6) relative to a corresponding control pig. “Reduced” indicates that <3% of the Gal antigen levels are present relative to a control pig. Heart tissue and heart valves from wild-type pigs are positive for the Gal antigen. See, FIGS. 1-3, respectively. Even commercially available porcine heart valve devices, which have undergone in vitro processing to reduce immunogenicity, have detectable Gal antigen (FIG. 4). Consequently, such xenografts will be rejected with time after implantation. Thus, using xenografts from α1-3 galactosyl transferase knockout pigs, which have reduced or no detectable Gal antigen, can further reduce the immunogenicity of the xenografts. Furthermore, processing of xenografts obtained from α1-3 galactosyl transferase knockout pigs or wild-type pigs can be monitored by detecting the level of the Gal antigen on the xenograft.

Pigs Having Disruptions in the α1-3 galactosyl Transferase Nucleic Acid Sequence

Nuclear transplantation can be used to generate transgenic pigs whose genomes contain a disruption of an endogenous α1-3 galactosyl transferase nucleic acid sequence. For example, fetal fibroblasts can be genetically modified such that they contain an inactivated endogenous α1-3 galactosyl transferase allele thereby preventing expression of an active α1-3 galactosyl transferase, and then fused with enucleated oocytes. After activation of the oocytes, the eggs are cultured to the blastocyst stage, and implanted into a recipient. See, Cibelli, et al., Science, (1998) 280:1256-1258, and Sharma, et al., Transplantation (2003) 75(4):430-436. Adult somatic cells, including, for example, cumulus cells and mammary cells, also can be used to produce pigs. See, for example, Wakayama, et al., Nature, (1998) 394(6691):369-374; and Wilmut, et al., Nature, (1997) 385(6619):810-813. Nuclei can be removed from genetically modified adult somatic cells, and transplanted into enucleated oocytes. After activation, the eggs can be cultured to the 2-8 cell stage, or to the blastocyst stage, and implanted into a suitable recipient. Wakayama, et al., 1998, supra. Transgenic pigs heterozygous for a disrupted endogenous α1-3 galactosyl transferase allele can be mated to produce homozygous pigs.

To determine if the Gal antigen is present on the surface of cells from the heterozygous or homozygous animals, tissue can be removed from the animal then embedded using, for example, OCT (TISSUE-TEK, Sakura) embedding medium. Tissues can be sectioned, placed on glass slides, air-dried, and stored at −80° C. until use. The sectioned tissues can be stained for the Gal antigen after fixing the sections in acetone, washing in water, blocking the slides, then incubating with the lectin GSIB4. GSIB4 is commercially available from, for example, Molecular Probes, Inc. (Eugene, Oreg.). The lectin can be labeled, either directly or indirectly. Suitable labels include, without limitation, radionuclides (e.g., ¹²⁵I, ¹³¹I, ³⁵S, ³H, ³²P, ³³P, or ¹⁴C), fluorescent moieties (e.g., fluorescein, FITC, PerCP, rhodamine, or PE), luminescent moieties (e.g., Qdot™ nanoparticles supplied by the Quantum Dot Corporation, Palo Alto, Calif.), compounds that absorb light of a defined wavelength, or enzymes (e.g., alkaline phosphatase or horseradish peroxidase). The lectin can be indirectly labeled by conjugation with biotin then detected with avidin or streptavidin labeled with a molecule described above. Methods of detecting or quantifying a label depend on the nature of the label and are known in the art. Examples of detectors include, without limitation, x-ray film, radioactivity counters, scintillation counters, spectrophotometers, colorimeters, fluorometers, luminometers, and densitometers. Combinations of these approaches (including “multi-layer” assays) familiar to those in the art can be used to enhance the sensitivity of assays.

### Obtaining Heart Valve Xenografts

As used herein, “heart valve xenograft” refers to pericardial tissue or to a heart valve (e.g., aortic, tricuspid, bicuspid, or pulmonary) or a portion of a heart valve (e.g., a leaflet), from transgenic pigs having a disruption of an α1-3 galactosyl transferase nucleic acid sequence. Heart valve xenografts can be obtained by removing an intact heart from the transgenic pig and excising suitable heart valve tissues. In some embodiments, a portion of a valve can be dissected such that it is free of adjoining tissue. For example, valves can be excised as separate leaflets. Alternatively, valves can be excised intact, including the fibrous ring surrounding the auriculo-ventricular orifice and the tendinous chords. Adhering tissue, plaques, calcifications and the like also can be removed. Porcine peritoneum or pericardium can be harvested using procedures known to the art. See, for example, the peritoneum harvesting procedure discussed in U.S. Pat. No. 4,755,593, herein incorporated by reference in its entirety.

It is particularly useful to collect the heart as soon as possible after slaughter of the animal. Typically, harvesting of the heart is performed in the cold, i.e., in the approximate range of about 5° C. to about 20° C., and under strict sterile technique to minimize damage to the heart tissue. The heart can be placed in a suitable sterile isotonic or other tissue preserving solution.

In some embodiments, the xenograft can be supported using stents, rings and the like. For example, two or three leaflets can be sewn to a generally circular supporting wire frame or stent. The wire frame or stent can provide a stable support structure for the valve leaflets, and impart a degree of controlled flexibility to reduce stress on the leaflet tissue during valve closure. A biocompatible cloth covering can be provided on the wire frame or stent to provide sewing attachment points for the leaflet commissures and cusps. Similarly, a cloth covered suture ring also can be attached to the wire frame or stent to provide an attachment site for sewing the valve structure in position within the patient's heart during implantation.

The xenograft can be prepared for implantation in a human using known techniques. See, for example, U.S. Pat. Nos. 6,383,732 and 6,102,944, herein incorporated by reference in their entirety. For example, the xenograft can be contacted with a fixative. Typically, this is performed to tan or crosslink the proteins within the extracellular components, to further diminish or reduce immunogenicity of the xenograft. Any fixative can be used for this treatment, and more than one fixing step can be performed or more than one fixative can be used. Suitable fixatives include, for example, gluteraldehyde, formaldehyde, adipic dialdehyde, an aliphatic diamine, an aromatic diamine, a carbodiimide, or a diisocyanate. Gluteraldehyde is particularly useful. For example, the xenograft can be contacted with a buffered solution containing from about 0.001% to about 5% gluteraldehyde (e.g., 0.1 to 5%) and having a pH of about 7.4. Any suitable buffer can be used, including phosphate buffered saline or trihydroxymethylaminomethane, that can maintain control over the pH for the duration of the fixation. Typically, fixation can be performed from one to 14 days (e.g., one to five or three to five days).

Alternatively, the xenograft can be exposed to a fixative in a vapor form, including, but not limited to, a vaporized aldehyde fixative, such as, for example, vaporized formaldehyde. For example, the xenograft can be exposed to a vaporized fixative having a concentration of about 0.001% to about 5.0% (e.g., about 0.01% to about 5.0%), and a pH of about 7.4. Exposure to a vaporized fixative can result in less residual chemicals in the xenograft.

After fixation, the xenograft can be rinsed to remove residual chemicals, and 0.01-0.1 M glycine (e.g., 0.01-0.05 M glycine) can be added to cap any unreacted aldehyde groups that remain.

In some embodiments, the xenograft can be subjected to a freeze/thaw cycle to kill the xenograft's cells. The xenograft can be frozen using any known method. For example, the xenograft can be dipped into liquid nitrogen or frozen slowly by placing it in a freezer. The xenograft can be thawed by immersion in an isotonic saline bath at room temperature (about 25° C.) for about 10 minutes.

The xenograft can be coated with anticalcification agents, antithrombotic coatings, antibiotics, growth factors, or other drugs that can enhance the incorporation of the xenograft into the recipient.

In some embodiments, the xenograft can be sterilized. For example, the xenograft can be sterilized using liquid systems (e.g., with gluteraldehyde and formaldehyde), ethylene oxide or propylene oxide, or radiation. In addition, a xenograft of the invention can be treated with polyethylene glycol (PEG), or treated with limited digestion by proteolytic enzymes such as ficin or trypsin to increase tissue flexibility.

The xenograft may be stored frozen until required for use. To freeze a xenograft, the xenograft can be contacted with a solution containing a cryoprotectant (e.g., dimethylsulfoxide (DMSO), glycerol, albumin, mono- and disaccharides, or serum such as fetal calf serum). For example, the solution can contain about 0 to 25% DMSO, 0 to 25% glycerol, or 0 to 50% albumin.

The heart valve xenograft can implanted into damaged human hearts by those of skill in the art using known surgical techniques, including, for example, open heart surgery, or minimally invasive techniques such as endoscopic surgery and transluminal implantation. Specific instruments for performing such surgical techniques are known to those of skill in the art.

### Articles of Manufacture

Xenografts can be combined with packaging materials and sold as articles of manufacture. Components and methods for producing articles of manufactures are well known. The articles of manufacture may combine one or more components described herein. For example, the xenograft can be packaged with a storage solution such as buffered saline, a tissue preservative, or a cryoprotectant, in a sterile container. In some embodiments, the xenograft is attached to a stent. Instructions describing how the xenograft can be used to treat a patient also can be included.

The invention will be further described in the following examples, which do not limit the scope of the invention described in the claims.

### EXAMPLE

The presence of the Gal antigen was assessed on heart valves from wild-type (i.e., no disruption in the α1-3 galactosyl transferase gene) and α1-3 galactosyl transferase knock-out pigs. Heart tissues and/or heart valves were dissected from the heart. Small portions of each were placed in OCT (TISSUE-TEK, Sakura) embedding medium and frozen at −80° C. For all samples, 5 micron sections were cut from frozen OCT embedded tissue and stained using standard immunohistological methods. Expression of the α-gal antigen (galactose α1,3 galactose β1,4 N-acetylglucosamine trisaccharide) was detected by binding of a horse radish peroxidase conjugated GSIB4 lectin (GSIB₄-HRP) and visualized using standard DAB staining. The specificity of lectin binding for the α-gal antigen was demonstrated by competitive inhibition using 10 mM α-gal trisaccharide sugar (GSIB₄-HRP+10 mM α-Gal sugar) to block lectin binding. The Gal antigen was not detectable in the heart of an α1-3 galactosyl transferase knockout pig (FIGS. 5 and 6), but was detectable in the heart and a heart valve from a wild-type pig (FIGS. 1-3).

Commercially available bioprosthetic porcine heart valves were sectioned and stained for the Gal antigen as described above. As indicated in FIG. 4, the Gal antigen was detectable on the commercially available device.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method of treating a patient, said method comprising implanting into said patient a porcine heart valve xenograft; wherein cells of said xenograft contain a disruption of the α1-3 galactosyl transferase nucleic acid sequence.

2. The method of claim 1, wherein said heart valve xenograft is a tricuspid valve or a portion thereof.

3. The method of claim 1, wherein said heart valve xenograft is a mitral valve or a portion thereof.

4. The method of claim 1, wherein said heart valve xenograft is an aortic valve or a portion thereof.

5. The method of claim 1, wherein said heart valve xenograft is a pulmonary valve or a portion thereof.

6. The method of claim 1, wherein said heart valve xenograft is pericardial tissue.

7. An article of manufacture comprising a porcine heart valve xenograft and a storage solution, wherein cells of said xenograft contain a disruption of the α1-3 galactosyl transferase nucleic acid sequence.

8. The article of manufacture of claim 7, wherein said storage solution is saline, a tissue preservative, or a cryoprotectant.

9. The article of manufacture of claim 8, wherein said cryoprotectant is dimethylsulfoxide, glycerol, albumin, monosaccharides, disaccharides, or serum.

10. A method of preparing a xenograft heart valve for implantation in a human, said method comprising providing a xenograft from a pig, wherein said xenograft comprises a portion of a heart valve, wherein said pig's genome comprises a disruption of an α1-3 galactosyl transferase nucleic acid sequence, said disruption resulting in endothelial cells of said pig having reduced or no detectable expression of Gal α1-3Galβ1-4GlcNac on their surface relative to cells of a control pig; and contacting said xenograft with a fixative.

11. The method of claim 10, wherein said fixative is selected from the group consisting of gluteraldehyde, formaldehyde, adipic dialdehyde, an aliphatic diamine, an aromatic diamine, a carbodiimide, and a diisocyanate.

12. The method of claim 10, wherein said fixative is gluteraldehyde.

13. The method of claim 10, wherein said method further comprises subjecting said xenograft to a freeze/thaw cycle.

14. The method of claim 10, wherein said method further comprises contacting said xenograft with an agent selected from the group consisting of an anti-calcification agent, an anti-thrombotic agent, an antibiotic, and a growth factor.

15. The method of claim 10, wherein said method further comprises sterilizing said xenograft.

16. An article of manufacture comprising a heart valve xenograft from a pig, wherein said pig's genome comprises a disruption of an α1-3 galactosyl transferase nucleic acid sequence, said disruption resulting in endothelial cells of said pig having reduced or no detectable expression of Gal α1-3Galβ1-4GlcNac on their surface relative to cells of a control pig.

17. The article of manufacture of claim 16, wherein said xenograft is attached to a stent.
